(19) United States
(12) Patent Application Publication
(10) Pub. No.: US 2003/0008393 A1
(43) Pub. Date: Jan. 9, 2003

(54) gammac negative hematopoietic progenitor cells

(75) Inventors: Gay M. Crooks, Sherman Oaks, CA (US); Robertson Parkman, Pasadena, CA (US); Kenneth Weinberg, South Pasadena, CA (US)

Correspondence Address: PERKINS COIE LLP, POST OFFICE BOX 1208, SEATTLE, WA, 98111-1208, US

(73) Assignee: Children's Hospital of Los Angeles

(21) Appl. No.: 10/136,855

(22) Filed: Apr. 29, 2002

Related U.S. Application Data
(63) Continuation of application No. PCT/US00/29143, filed on Oct. 20, 2000.
(60) Provisional application No. 60/162,348, filed on Oct. 29, 1999.

(51) Int. Cl. C12N 5/08

(52) U.S. Cl. 435/372

(57) ABSTRACT

Methods of purifying cell suspensions and cell suspensions containing hematopoietic progenitor cells, but not leukemic cells, are provided, as well as therapeutic methods employing the cell suspensions.

### RELATED APPLICATIONS

This application claims priority to the U.S. Provisional Application Serial No. 60/162,348, filed Oct. 29, 1999, by Crooks, et al., and entitled “γc Negative Hematopoietic Progenitor Cells,” which is incorporated herein by reference in its entirety, including any drawings.

### GOVERNMENT LICENSE RIGHTS

The U.S. Government has a paid-up license in this invention and the right in limited circumstances to require the patent owner to license others on reasonable terms as provided for by the terms of Grant No. A140581 awarded by NIH.

### FIELD OF INVENTION

The present invention is directed to the isolation of normal hematopoietic progenitor cells.

### BACKGROUND OF THE INVENTION

Hematopoietic stem cells (HSC) are the cells which give rise to all cellular blood elements, including red blood cells, granulocytes, monocytes, dendritic cells, platelets and lymphocytes. Transplantation of HSC is an effective means of treating a variety of hematological, immunologic and oncologic disorders. For patients with cancer who do not have an HLA-matched donor, autologous transplantation of HSC is possible if either the tumor does not involve the marrow or peripheral blood or if there is a method to remove the malignant cells from the marrow or peripheral blood, while sparing the stem cells.

For acute leukemia, for example, methods to purge leukemia cells from autologous marrow have depended either on the in vitro use of chemotherapeutic drugs or on immunologic methods to remove or kill the leukemic cells. A difficulty with the chemotherapeutic approach has been non-specific toxicity to the normal HSC as well as drug resistance in the leukemic cells.

Immunologic methods depend on differences in expression of surface antigens or markers which can then be used to either target the leukemic cells or to allow physical separation of the normal and leukemic cells. It has been found, however, that many of the surface markers which have been used to isolate HSC do not discriminate between normal and leukemic cells. CD34, for example, is a glycoprotein which is present on normal HSC, but is also expressed at high frequency by leukemic cells. CD38, a different surface glycoprotein, is not expressed by HSC, but is frequently expressed by leukemic cells. See, e.g., Lamkin, et al., Immunophenotypic differences between putative hematopoietic stem cells and childhood B-cellprecursor acute lymphoblastic leukemia cells, Leukemia 8(11):1871-8 (1994).

Cell markers presently used for the selection of HSC in some cases cannot distinguish normal HSC from unwanted (for example malignant) cells. Malignant cells, for example leukemic cells from some patients with either acute non-lymphoblastic leukemia (ANLL) or acute lymphoblastic leukemia (ALL) have been shown to be CD34+ CD38−. Bonnet, et al., Human acute myeloid leukemia is organized as a hierarchy that originates from a primitive hematopoietic cell, Nat Med 3(7):730-7 (1997) and Example III, infra. Leukemic cells therefore may be present in CD34+CD38− populations, and will not be excluded when HSC are isolated using these markers.

The common gamma chain (γc) of the interleukin-2 receptor is the product of the IL2RG gene. See, e.g., Takeshita, et al., Cloning of the gamma chain of the human IL-2 receptor, Science 257(5068):379-82 (1992); Sugamura, et al., The common gamma-chain for multiple cytokine receptors, Adv Immunol. 59:225-77 (1995). γc is a cell surface protein expressed during hematopoietic differentiation. Noguchi M, et al., Interleukin-2 receptor gamma chain mutation results in X-linked severe combined immunodeficiency in humans, Cell 73(1):147-57 (1993); Uribe, et al., X-linked SCID and other defects of cytokine pathways, Semin Hematol. 35(4):299-309 (1998). The γc gene has also been shown to be a shared component of IL-4, IL-7, IL-9 and IL-15 receptors.

It would be desirable, therefore, to improve the current methods and markers used for isolating HSC cells.

### SUMMARY OF THE INVENTION

It is an object of the present invention to provide a suspension of cells having pluripotent hematopoietic stem cells substantially free of γc+ cells.

Another object of the present invention is to provide therapeutic methods employing a suspension of cells having pluripotent hematopoietic stem cells substantially free of γc+ cells.

Another object of the present invention is to provide antibodies that distinguish undesired cells, that otherwise express pluripotent hematopoietic stem cells markers, from normal hematopoietic progenitor cells.

Another object of the present invention is to provide a method for preparing a cell population useful for stem cell transplantation that is enriched in pluripotent hematopoietic stem cells substantially free of γc+ cells.

Still another object of the present invention is to provide a therapeutic method of autologous stem cells transplantation for individuals undergoing treatment for leukemia.

### DETAILED DESCRIPTION OF THE INVENTION

In one embodiment of the present invention, it has been discovered that a cell suspension having hematopoietic stem cells not expressing the common gamma chain contains most of the pluripotent progenitors and almost all the slowly dividing, cytokine resistant progenitors. Cell populations purified in accordance with the invention therefore are highly enriched with human hematopoietic progenitors.

In another embodiment of the present invention, it has been discovered that a purified normal hematopoietic progenitor cell suspension free of γc+ malignant cells, in particular γc+ leukemia cells, may be obtained from a sample derived from an individual affected by such malignancy by purifying the cells in accordance with the present invention.

Any cell suspension having the desired cells may be used for the preparation of the purified cells of the present invention. Preferably the cell suspensions are obtained from bone marrow, blood and/or umbilical cord. For peripheral blood stem cell collection, see, e.g., Petris M G, et al., Peripheral blood stem cell collection and transplantation in paediatric malignancies: a monocentric experience, Bone Marrow Transplant. 22 Suppl 5:S13-5 (1998); Marson, et al., Collection of a peripheral blood stem cells in pediatric patients: a concise review on technical aspects, Bone Marrow Transplant. 22 Suppl 5:S7-11 (1998). When peripheral blood is utilized, hematopoietic stem cells may be mobilized prior to the apheresis procedure, or any other procedure that may be used for collection. Any effective mobilization technique may be used. See, e.g. Shpall, The utilization of cytokines in stem cell mobilization strategies, Bone Marrow Transplant. 23 Suppl 2:S13-9 (1999).

In accordance with the present invention, the cells are substantially purified based on satisfying at least two criteria. First, that the cells represent hematopoietic progenitor or stem cells. Second, that the cells are γc−.

As used herein, the term “hematopoietic stem cell” is used to describe the subset of “hematopoietic progenitor cells,” and means cells that can produce both mature hematopoietic and lymphoid cells. Any effective method may be used to purify the hematopoietic stem cells. If the hematopoietic stem cells are purified using a marker, any effective marker or panel of markers may be used.

“Stem cells” are formative cells whose daughter cells may give rise to other cell types. Stem cells may divide symmetrically, i.e., give rise to two identical stem cells, or divide asymmetrically, which would then produce one determined cell and one stem cell. “Unipotent stem cells” give rise to one end-stage differentiated cell, whereas “pluripotent stem cells” give rise to two or more independent types of differentiated cells.

Methods and apparatus for the separation of cells based on the presence or absence of a specific known marker are well known in the art. Any such method may be used in accordance with the present invention. A few, non exclusive methods are described in the following articles: Thomas, et al., Purification of hematopoietic stem cells for further biological study, Methods 17(3):202-18 (1999); Papadimitriou, et al., Immunomagnetic selection of CD34+ cells from fresh peripheral blood mononuclear cell preparations using two different separation techniques, J Hematother. 4(6):539-44 (1995); Steinitz M, et al., Separation of rare cell subpopulations with the aid of biotin-labeled ligands, Med Oncol Tumor Pharmacother. 10(1-2):49-52 (1993); Chalmers J J, et al., Theoretical analysis of cell separation based on cell surface marker density, Biotechnol Bioeng. July 5, 1998;59(1):10-20; Sun L, et al., Continuous, flow-through immunomagnetic cell sorting in a quadrupole field, Cytometry. Dec. 1, 1998;33(4):469-75; Partington K M, et al., A novel method of cell separation based on dual parameter immunomagnetic cell selection, J Immunol Methods. Mar. 4, 1999;223(2): 195-205; Schmitz B, et al., Magnetic activated cell sorting (MACS)—a new immunomagnetic method for megakaryocytic cell isolation: comparison of different separation techniques, Eur J Haematol. 1994 May;52(5):267-75; Zimmerman T M, et al., Large-scale selection of CD34+ peripheral blood progenitors and expansion of neutrophul precursors for clinical applications, J Hematother. 1996 June;5(3):247-53; Beaujean, Methods of CD34+ cell separation: comparative analysis, Transfus Sci. 1997 June;18(2):251-61; Crooks G M, et al., Constitutive HOXA5 expression inhibits erythropoiesis and increases myelopoiesis from human hematopoietic progenitors, Blood. Jul. 15, 1999;94(2):519-28.

The purity of the cells obtained by practicing the present invention may of course vary depending on the limitations of the techniques and apparatus used. The cells are preferably as pure as possible. In particular, the cells are preferable at least 85% pure, more preferably at least about 90% pure.

In one preferred embodiment, purified progenitor cells are obtained from a sample based on the presence of the CD34 antigen (CD34+cells). Other markers, or additional markers may also be used, for example Thy-1+, CD38−, CD33− and CD13−. Most preferably the purified progenitor cells are CD34+ and CD38−.

The second criterion is satisfied by purifying cells based on the absence of the marker of the present invention, the “common gamma chain” or γc. As used herein, γc is the common gamma chain of the interleukin-2 receptor. See, e.g., Takeshita, et al., Cloning of the gamma chain of the human IL-2 receptor, Science 257(5068):379-82 (1992); Sugamura, et al., The common gamma-chain for multiple cytokine receptors, Adv Immunol. 59:225-77 (1995). The cells may also be purified based on the absence of the IL-2, IL-4, IL-7, IL-9, and/or IL-15 receptors, of which γc is known to form a part. Effective combinations of these markers may also be used.

As used herein, “+” and “−” mean that the specific marker is present or absent, respectively, as commonly used in the art.

The purification of cells based on each criterion may be performed separately, i.e. initially purifying CD34+ cells, for example, see, e.g., U.S. Pat. Nos. 4,714,680 and 4,965,204, then further purifying this population based on the absence of the marker of the present invention, or vice-versa. Alternatively, the purification is made in a single step.

In another aspect, purified cells obtained in accordance with the present invention include preferably higher than about 10% of single cells having both lymphoid and myeloid potential (pluripotentiality). More preferably at least about 12% of single cells have both lymphoid and myeloid potential. Most preferably at least about 13.5% of single cells have both lymphoid and myeloid potential.

The generative capacity is established by CFU production after stromal co-cultivation. Lymphoid potential is analyzed by FACS identification of B lymphoid i dendritic and NK cells. Myeloid potential is analyzed as generation of CFU.

In another aspect, the purified cells obtained in accordance with the present invention have preferably higher than about 5% cells that proliferated late, i.e. after day 28 in the Extended Long Term Culture-Initiating Cell (ELTC-IC) assay. More preferably at least about 5.5% cells proliferated late in the ELTC-IC assay. Most preferably at least about 6.5% cells proliferated late in the ELTC-IC assay.

The assays are well known in the art and described in the literature. See, e.g., Case S S, et al., Stable transduction of quiescent CD34(+)CD38(−) human hematopoietic cells by HIV-1-based lentiviral vectors, Proc Natl Acad Sci U S A. Mar. 16, 1999;96(6):2988-93; Fluckiger A C, et al., In vitro reconstitution of human B-cell ontogeny: from CD34(+) multipotent progenitors to Ig-secreting cells, Blood. Dec. 15, 1998;92(12):4509-20; Thiemann F T, et al., The murine stromal cell line AFT024 acts specifically on human CD34+CD38 progenitors to maintain primitive function and immunophenotype in vitro, Exp Hematol. 1998 July;26(7):612-9; Hao Q L, et al., In vitro identification of single CD34+CD38− cells with both lymphoid and myeloid potential, Blood. Jun. 1, 1998;91(11):4145-51; Dao M A, et al., Engraftment and retroviral marking of CD34+ and CD34+CD38−human hematopoietic progenitors assessed in immune-deficient mice, Blood. Feb. 15, 1998;91(4):1243-55; Rawlings D J, et al., Differentiation of human CD34+CD38− cord blood stem cells into B cell progenitors in vitro, Exp Hematol. 1997 January;25(1):66-72; Yates K E, et al., Analysis of Fes kinase activity in myeloid cell growth and differentiation, Stem Cells. 1996 November; 14(6):714-24; Hao Q L, et al Extended long-term culture reveals a highly quiescent and primitive human hematopoietic progenitor population, Blood. Nov. 1, 1996;88(9):3306-13; Shah A J, et al., Flt3 ligand induces proliferation of quiescent human bone marrow CD34+CD38− cells and maintains progenitor cells in vitro, Blood. May 1, 1996;87(9):3563-70; Hao Q L, et al., A functional comparison of CD34 +CD38− cells in cord blood and bone marrow, Blood. Nov. 15, 1995;86(10):3745-53.

Another application of the present invention is the isolation of a highly enriched source of stem cells free from malignant cells. The cells thus isolated may be used for human hematopoietic stem cell transplantation, for example, significantly decreasing the risk of relapse from contamination by residual malignant cells when autologous reinfusionu is being used. The cell are therefore useful in the therapeutic procedures of γc+ leukemias or other malignancies.

The use of hematopoietic stem cells for the reconstitution of the lympho-hematopoietic system is well known in the art. See, e.g., Parkman R, et al., Immunological reconstitution following bone marrow transplantation, Immunol Rev. 1997 June; 157:73-8; Kapoor N, et al., Hematopoietic stem cell transplantation for primary lymphoid immunodeficiencies, Semin Hematol. 1998 October;35(4):346-53; Appelbaum F, et al., American Society for Blood and Marrow Transplantation guidelines for training, Biol Blood Marrow Transplant. 1995 November;1(1):56; Parkman R, et al., Bone marrow transplantation for metabolic diseases, Cancer Treat Res. 1995;76:87-96; Thomas, A history of haemopoietic cell transplantation, Br J Haematol. 1999 May;105(2):330-9.

The present invention provides a significant advance in the art of normal hematopoietic progenitor cell purification. Although the γc is expressed on mature hematopoietic cells and on most progenitor cells, it is not expressed by hematopoietic stem cells. While transplantation of stem cells has the ability to restore the production of hematopoietic and lymphoid cells to a patient who has lost such production due to, for example, radiation therapy or high dose chemotherapy, present methods of purification are not always adequate to separate normal hematopoietic progenitor cells from malignant cells.

Unlike other antigens known to date, the marker disclosed herein is not expressed by hematopoietic stem cells, but is expressed in most malignant cells of hematopoietic origin studied to date, in particular leukocytes. The marker is also expressed in mature myeloid and lymphoid cells. The present invention also provides antibodies which facilitate the isolation of the desired cells and make possible improved therapeutic techniques that prevent transplantation of hematopoietic stem cells contaminated with malignant cells.

The present invention contemplates any method employing effective anti-(γc) antibodies to separate normal hematopoietic progenitor cells from malignant (γc) expressing cells, in particular those of hematopoietic origin.

The stem cells isolated in accordance with the present invention can also be employed to produce panels of monoclonal antibodies to stem cells. The stem cells can also be cultivated and/or genetically modified. Genetically modified hematopoietic stem cells may be used for gene therapy, for example. See, e.g., Lutzko, et al., Recent progress in gene transfer into hematopoietic stem cells, Crit Rev Oncol Hematol. 30(2): 143-58 (1999); Kume, et al., Hematopoietic stem cell gene therapy: a current overview, Int J Hematol. Jun;69(4):227-33 (1999); Moore M A, et al., Optimizing conditions for gene transfer into human hematopoietic cells, Prog Exp Tumor Res. 36:20-49 (1999); Sadelain M, et al., Basic principles of gene transfer in hematopoietic stem cells, Prog Exp Tumor Res. 36:1-19 (1999); Zanjani, Prospects for in utero human gene therapy, Science 285(5436):2084-8 (1999); Sirchia, et al., Placental/umbilical cord blood transplantation, Haematologica 84(8):738-47 (1999); Surbek, et al., In utero hematopoietic stem cell transfer: current status and future strategies, Eur J Obstet Gynecol Reprod Biol. 85(1):109-15 (1999); Mascarenhas L, et al., Gene delivery to human B-precursor acute lymphoblastic leukemia cells, Blood. Nov. 15, 1998;92(10):3537-45; Weinberg K I, et al., Gene therapy for congenital lymphoid immunodeficiency diseases, Semin Hematol. 1998 October;35(4):354-66; Uribe L, et al., X-linked SCID and other defects of cytokine pathways, Semin Hematol. 1998 October;35(4):299-309; Kohn D B, et al., Engraftment of gene modified umbilical cord blood cells in neonates with adenosine deaminase deficiency, Nat Med. 1995 October;1(10):1017-23.

The anti-(γc) antibodies of the present invention can be produced by any effective means readily available to those of skill in the art. Preferred are anti-(γc) monoclonal antibodies. Any effective technique may be used to produce the immortal, monoclonal antibody-secreting cell line. The general methodology for making monoclonal antibodies is well known to the art. See, e.g., M. Schreier et al., Hybridoma Techniques (Cold Spring Harbor Laboratory 1980); Hammerling et al., Monoclonal Antibodies and T-Cell Hybridomas (Elsevier Biomedical Press 1981); Kennett et al., Monoclonal Antibodies (Plenum Press 1980); Malek T R, et al., J Leukoc Biol. 63(6):643-9 (1998).

A preferred hybridoma producing a monoclonal anti-γc antibody is produced by challenging a mouse with the γc protein, or portion thereof, and fusing the recovered B-lymphocytes with an immortal mouse plasmacytoma cell, or the like. Antibody-producing immortal cells can be screened for anti-(γc) antibody production by any means available in the art. For example, by selecting clones that are strongly reactive with cells expressing the γc marker, but not reactive with cells not expressing the marker, or their ability to bind γc or an appropriate epitopes in an ELISA like assay, a western blot, or the like. The hybridoma producing monoclonal anti-(γc) antibody γc1 is preferred.

Antibodies produced by clones which show those properties can then be screened for the additional properties of labeling leukemic and mature hematopoietic cells but not normal hematopoietic progenitor cells. Monoclonal antibodies corresponding to monoclonal anti-(γc) antibody γc1 are preferred, and the monoclonal anti-(γc) antibody γc1 is particularly preferred. One antibody corresponds to another antibody if they both recognize the same or overlapping antigen binding sites as demonstrated by, for example, a binding inhibition assay, or the like.

The hematopoietic progenitor cells purified based on the additional fact of the absence of a γc marker can be used as an immunogen, as described above, to produce a panel of monoclonal antibodies against stem cells and immature marrow cells. The production of anti-stem cell antibodies which do not recognize malignant cells is greatly facilitated by the use of substantially pure populations of non-malignant hematopoietic precursor cells. The specificities of such antibodies can be determined readily through routine screening by one skilled in the art. Thus, non-malignant, hematopoietic progenitor cell antigens (and antibodies to these antigens) can be identified by those of skill in the art.

Reagents and other components necessary for performing the methods of the present invention may be assembled into a package or the like, which is also considered novel.

All the references and patents cited herein are hereby incorporated by reference in their entirety, including figures, as if fully set forth herein.

### EXAMPLES

The following examples are provided to illustrate specific embodiments of the present invention. The examples are included for illustrative purposes only and are not intended to limit the scope of the present invention.

### Example I

Generation of γc1 Monoclonal Antibody.

Balb/c mice were immunized with normal human lymphoid cells. Spleen cells were harvested for hybridoma generation. The supernatants from the hybridomas were screened to determine which ones reacted with γc. Screening was performed by enzyme linked immunoassay (ELISA) directed against either a viable murine B lymphocyte cell line or a murine cell line transfected with the gene encoding the normal γc gene. The use of viable cells in the ELISA screening assured that only antibodies reactive with extracellular epitopes of γc would be selected. Wells reactive with the human γc were then expanded and subcloned by limiting dilution to isolate a hybridoma which produced a monoclonal antibody termed “γc1”. The γc1 antibody was demonstrated to react with γc expressed on the cell surface by flow cytometric analyses. The γc1 antibody was conjugated with phycoerithritin (PE) and allophallocyanin (APC).

### Example II

Isolation of CD34+CD38− γc− Cells.

CD34+CD38− γc− progenitors can be isolated from bone marrow or umbilical cord blood cells. The CD34+ cells can be enriched first with an immunomagnetic bead column. Alternatively, CD34+CD38− γc− cells can be isolated directly from the stem cells source without pre-enrichment for CD34+ cells. The CD34+CD38− γc− cells can then be isolated by staining the CD34+ cells with directly labeled antibodies, typically anti-CD34 antibody bound to fluorescein isothiocyanate (FITC), anti-CD38 antibody bound to PE, and the γc1 antibody bound to APC. CD34+CD38− γc− cells can then be isolated by flow cytometry. Isotype control antibodies bound to the respective fluorochromes are used to determine the fluorescence intensity of positive and negative staining. Using this information, sort gates would then be generated for isolation of the CD34+CD38− γc− cells. Typically, the CD34+CD38− cells would have the greatest intensity of CD34 staining and would have CD38 staining which is less than that of half the mean channel number of the isotype control. The γc staining of the γc− fraction of the cells is also typically less than one-half of the isotype control staining. The sorted cells can then be analyzed for purity by re-staining, or for pluripotentiality and self-renewal by in vitro culture, e.g., extended long term culture initiating cell assays) or transfer into immunodeficient mice.

### Example III

Sorting of CD34+CD38− γc− Cells.

Functionally Heterogeneous CD34+CD38− Population of Cells Includes a Subpopulation of Pluripotent Progenitor Cells.

The CD34+CD38− immunophenotype defines a population of human hematopoietic progenitors that is functionally heterogeneous in terms of cytokine responsiveness, generative capacity and lineage potential. The present experiment describes a method of enriching the functionally distinct subpopulations of CD34+CD38− cells with cells including pluripotent progenitors, slow dividing, cytokine resistant progenitors (ELTC-IC).

Most lineage specific antigens are not expressed on CD34+CD38− cells. We have discovered that the common gamma chain (γc), a component of the receptors for IL-2, IL-4, IL-7, IL-9, and IL-15, is detectable by FACS on the surface of 41.5±5.9% of cord blood CD34+CD38− cells.

### Example IV

Morphologic and Cytochemical Phenotype of CD34+CD38− γc− Cells.

Extended Long Term Culture-Initiating Cell (ELTC-IC) Assay and Lympho-Myeloid Switch Culture Assay of CD34+CD38− Cell Subpopulations.

Functional analysis in the ELTC-IC assay revealed that the most primitive progenitors were found in the CD34+CD38− cells which do not express γc (CD34+CD38− γc− cells). Overall cloning efficiency of CD34+CD38− γc− cells was significantly higher than that of CD34+CD38− γc+ cells in both myeloid and lymphoid assays (P=0.0006, n=4).

Generative capacity (CFU production after stromal co-cultivation) was higher in CD34+CD38− γc− cells than CD34+CD38− γc+ cells (P=0.0045). In our assays in general, about 9% of single CD34+CD38− have both lymphoid and myeloid potential. Lymphoid potential in these experiments was analyzed by FACS identification of B lymphoid±dendritic and NK cells. Myeloid potential was analyzed as generation of CFU.

We have discovered that further selection of this cell population for cells that do not express the γc marker (CD34+CD38− γc− cells) significantly increased to 13.9±0.3% the proportion of single cells having both lymphoid and myeloid potential. Only 1.8±1.3% of CD34+CD38− γc+ single cells had both lymphoid and myeloid potential.

ELTC-IC were found almost exclusively in the CD34+CD38− γc− cell population. In our assays in general, about 4% of CD34+CD38− cells proliferate late, i.e. after day 28. This proportion was significantly increased, to 6.5±2.5%, when the CD34+CD38− cell population was further selected for cells that do not express the γc marker (CD34+CD38− γc− cells). In contrast, only 0.3±0.3% CD34+CD38− γc+ cells proliferated late (P=0.036).

We have therefore identified a subset of CD34+CD38− cells (CD34+CD38− γc− cells) that contains most of the pluripotent progenitors and almost all the slowly dividing, cytokine resistant progenitors. The subpopulation may be immunophenotypically identified by lack of expression of γc, permitting the substantial purification of a population of cells highly enriched in the most primitive human hematopoietic progenitors.

### Example V

Expression of γc on Normal and Malignant Cells of Hematopoietic Origin.

Leukemic Cells are Present in CD34+CD38− Cells of Children Affected With ALL.

Previous studies showed that greater than 95% of childhood B lineage ALL express CD38. This study was carried out to evaluate the feasibility of isolating a pure population of normal hematopoietic progenitor cells from children with B lineage ALL by positive selection of CD34+CD38− cells.

CD34+ cells from bone marrow samples from 12 children with B lineage ALL were isolated at day 28 of treatment, when clinical remission had been attained. The CD34+ progenitor cells were flow cytometrically sorted into CD34+CD38+ and CD34+CD38− populations. The cells were then analyzed for the presence of clonotypic rearrangements of the TCR Vδ2-Dδ3 locus. Only patients whose diagnostic marrow had an informative TCR Vδ2-Dδ3 rearrangement were included in the study. The TCR Vδ2-Dδ3 rearrangements were detected by PCR amplification and Southern hybridization to an oligonucleotide probe derived from the sequence of the TCR Vδ2-Dδ3 N-region from the patient's diagnostic bone marrow sample. Detection thresholds were typically 10⁻⁴ to 10⁻⁵ leukemic cells. The absolute numbers of CD34+CD38− cells which could be obtained from 5 ml samples of Dδ8 bone marrow ranged from 16 to 3300.

In 9 out of 12 samples analyzed, the sorted CD34+CD38− cells had no detectable Vδ2-Dδ3 rearrangements. In 5 of these negative cases, the lower limit of detection was close to the number of cells obtained, leaving open the possibility that leukemic cells were present at a level below the limit of detection. In 3 cases, the clonotypic Vδ2-Dδ3 rearrangement was detected in the CD34+CD38− population, indicating that the putative normal HSC population also contained leukemic cells.

These results indicate that although most childhood ALL cells express CD34 and CD38, leukemic cells are also present in the CD34+CD38− population. Therefore, in the case of children with ALL, positive selection of CD34+CD38− cells in many instances will be inadequate for isolating normal hematopoietic progenitor cells for transplantation.

### Example VI

Leukemic Cells can be Immunophenotypically Distinguished and Separated from Normal HSC.

B-precursor ALL cells usually express the pan-hematopoietic cell antigen CD38 (>95%), the lineage specific B-lymphoid antigen CD19, and frequently the hematopoietic progenitor cell antigen CD34 (approximately 70%). Normal HSC are CD34+CD38−. However, this population also frequently contains leukemic cells in children with ALL. See Example above.

We discovered human B-precursor ALL cells expressing γc and IL-7Rα. The pattern of expression of CD34, CD38, γc, IL-7Rα and CD19 was analyzed on 39 consecutive B-precursor ALL bone marrow samples from newly diagnosed and relapsed patients. A leukemic population of cells was gated by flow cytometry based on staining with anti-CD45 PerCP, side scatter, and CD19 expression. These cells were then analyzed for co-expression of CD34, CD38, γc and IL-7Rα.

We discovered that 97% of the CD19+ leukemia samples co-expressed γc, 72% co-expressed IL-7Rα, and 100% co-expressed either γc or IL-7Rα. In all leukemia samples, γc and IL-7Rα were expressed in a unimodal distribution. The relative amount of γc expression was greater than that of IL-7Rα with mean fluorescent intensity (MFI) for γc of 163±129 and for IL-7Rα of 76±54.

γc and/or IL-7Rα expression on B-precursor ALL cells can be used to immunophenotypically distinguish leukemic cells from normal HSC. Normal HSC useful in autologous transplantation therefore can be isolated and separated from leukemic cells in samples derived from individuals affected with ALL by positive selection of CD34+CD38− (γc− and/or IL-7Rα−) cells.

Since variations will be apparent to those skilled in the art, it is intended that this invention be limited only by the scope of the appended claims.

What is claimed is:

1. A suspension of cells comprising pluripotent hematopoietic stem cells substantially free of γc+ cells.

2. The cell suspension of claim 1 wherein the pluripotent hematopoietic stem cells comprise single cells having both lymphoid and myeloid potential.

3. The cell suspension of claim 2 wherein greater than about 10% of single cells comprise pluripotent hematopoietic stem cells having both lymphoid and myeloid potential.

4. The cell suspension of claim 2 wherein greater than about 5% of cells comprise pluripotent hematopoietic stem cells that proliferated late as measured by the Extended Long Term Culture-Initiating Cell (ELTC-IC) assay.

5. The cell suspension of claim 1 wherein the pluripotent hematopoietic stem cells comprise CD34+ cells.

6. The cell suspension of claim 5 comprising greater than about 85% CD34+ γc− cells.

7. The cell suspension of claim 6 comprising greater than about 85% CD34+CD38− γc− cells.

8. The cell suspension of claim 6 comprising greater than about 85% CD34+CD38− γc− IL-7Rα− cells.

9. A method of purifying hematopoietic stem cells comprising
(a) obtaining a sample of cells that includes hematopoietic stem cells;
(b) separating the cells in the sample into CD34+ and CD34− cells;
(c) collecting the CD34+ cells;
(d) separating the CD34+ cells into CD34+ γc− and CD34+ γc+ cells;
(e) collecting the CD34+ γc− cells.

10. The method of claim 9 wherein (d) and (e) are modified as follows
(d) separating CD34+ cells into a first group of CD34+ γc− IL-7Rα− cells and a second group of CD34+ γc+ IL-7Rα−, CD34+ γc− IL-7Rα+ and CD34+ γc+ IL-7Rα+ cells;
(e) collecting the first group of cells.

11. A suspension of human cells from marrow or blood comprising CD34+ γc− cells and substantially free of cells that are not CD34+ γc−, said suspension having the ability to restore the production of lymphoid and hematopoietic cells to a human lacking said production.

12. A suspension of normal human cells derived from blood or bone marrow of an individual affected with leukemia comprising normal pluripotent hematopoietic stem cells substantially free of γc+ leukemia cells, mature lymphoid cells and mature myeloid cells.

13. The suspension of normal human cells of claim 12, wherein the normal pluripotent hematopoietic stem cells are substantially free of CD34+ γc+ IL-7Rα−, CD34+γc− IL-7Rα+ and CD34+ γc+ IL-7Rα+ leukemia cells, mature lymphoid cells and mature myeloid cells.

14. A method of purifying normal pluripotent hematopoietic stem cells substantially free of malignant cells from an individual having a γc+ malignancy comprising
(a) obtaining a sample of cells that includes hematopoietic stem cells from an individual having a γc+ malignancy;
(b) separating the cells in the sample into CD34+ and CD34− cells;
(c) collecting the CD34+ cells;
(d) separating the CD34+ cells into CD34+ γc− and CD34+ γc+ cells;
(e) collecting the CD34+ γc− cells.

15. The method of claim 14 wherein the individual has a γc+ IL-7Rα+ malignancy, wherein (d) and (e) are modified as follows
(d) separating CD34+ cells into a first group of CD34+ γc− IL-7Rα− cells and a second group of CD34+ γc+ IL-7Rα−, CD34+ γc− IL-7Rα+ and CD34+ γc+ IL-7Rα+ cells;
(e) collecting the first group of cells.

16. The method of claim 14, wherein the malignancy is leukemia.

17. The method of claim 16, wherein the leukemia is selected from the group consisting of acute non-lymphoblastic leukemia (ANLL) or acute lymphoblastic leukemia (ALL).

18. A kit for purifying hematopoietic stem cells, said kit having a package comprising
a means for selecting CD34+ cells, said means selected from the group consisting of labeled anti-CD34 antibodies, for labeling cells for separation by a mechanical cell sorter that detects the presence of the label, and anti-CD34 antibodies attached to a first solid support;
a means for selecting γc− cells, said means selected from the group consisting of labeled anti-γc antibodies, for labeling cells for separation by a mechanical cell sorter that detects the presence of the label, and anti-γc antibodies attached to a second solid support.

19. The kit of claim 18 wherein the first solid support is selected from the group consisting of agarose beads, polystyrene beads, magnetic beads, hollow fiber membranes and plastic petri dish.

20. The kit of claim 18 wherein the second solid support is selected from the group consisting of agarose beads, polystyrene beads, magnetic beads, hollow fiber membranes, plastic support and plastic petri dish.

21. The kit of claim 18 wherein the anti-CD34 antibodies are labeled with a fluorescent label or a magnetic label.

22. The kit of claim 18 wherein the anti-γc antibodies are labeled with a fluorescent label or a magnetic label.

23. The kit of claim 18 wherein the anti-γc antibodies are monoclonal antibodies.

24. The kit of claim 23 wherein the anti-γc antibodies are γy1 monoclonal antibodies.

25. A kit for purifying normal pluripotent hematopoietic stem cells substantially free of malignant cells from an individual having a γc+ malignancy comprising, said kit having a package comprising
a means for selecting CD34+ cells, said means selected from the group consisting of labeled anti-CD34 antibodies, for labeling cells for separation by a mechanical cell sorter that detects the presence of the label, and anti-CD34 antibodies attached to a first solid support;
a means for selecting γc− cells, said means selected from the group consisting of labeled anti-γc antibodies, for labeling cells for separation by a mechanical cell sorter that detects the presence of the label, and anti-γc antibodies attached to a second solid support.

26. The kit of claim 25 wherein the first solid support is selected from the group consisting of agarose beads, polystyrene beads, magnetic beads, hollow fiber membranes and plastic petri dish.

27. The kit of claim 25 wherein the second solid support is selected from the group consisting of agarose beads, polystyrene beads, magnetic beads, hollow fiber membranes, plastic support and plastic petri dish.

28. The kit of claim 25 wherein the anti-CD34 antibodies are labeled with a fluorescent label or a magnetic label.

29. The kit of claim 25 wherein the anti-γc antibodies are labeled with a fluorescent label or a magnetic label.

30. The kit of claim 25 wherein the anti-γc antibodies are monoclonal antibodies.

31. The kit of claim 30 wherein the anti-γc antibodies are γc1 monoclonal antibodies.

32. A method of reconstituting the lympho-hematopoietic system of an individual in need thereof comprising administering an effective amount of the cell suspension of claim 1.

33. A method of reconstituting the lympho-hematopoietic system of an individual in need thereof comprising administering an effective amount of the cell suspension of claim 12.
